(19) United States
(12) Patent Application Publication
(10) Pub. No.: US 2012/0003262 A1
(43) Pub. Date: Jan. 5, 2012

(54) ANTICANCER HEALTH FOOD CONTAINING PHELLINUS LINTEUS AND VEGETABLE WORMS

(75) Inventors: Ki Hwan Bae, Daejeon (KR); Wen Yi Jin, Daejeon (KR); Jae Kuk Yoo, Daejeon (KR); Byeong Wook Park, Daejeon (KR); Jin Pyo Kim, Daejeon (KR); Ick Soo Lee, Gumi-si (KR)

(73) Assignee: HAN KOOK SHIN YAK CO., LTD., Nonsan-si, Chungcheongnam-do (KR)

(21) Appl. No.: 13/142,963

(22) PCT Filed: Apr. 1, 2009
(86) PCT No.: PCT/KR2009/001666

§ 371 Date: Sep. 20, 2011

(30) Foreign Application Priority Data
Dec. 31, 2008 (KR) 10-2008-0137733

(51) Int. Cl. A61K 36/07; A61P 35/00

(52) U.S. Cl. 424/195.15

(57) ABSTRACT

Disclosed is a health food containing extracts of Phellinus linteus and vegetable worms for preventing and treating cancer. The health food contains ethanol extract of the mixture containing the mycelia of the Phellinus linteus and vegetable worms at a ratio of 1:5 to 5:1. The disclosed anticancer health food can enhance immunity and prevent cancer through the suppression and propagation suppression of cancer cells themselves. The suppression and reproduction rate suppression of the cancer cells themselves are achieved through the ingestion of the health food containing the Phellinus linteus and vegetable worms. Therefore, the disclosed anticancer health food can provide ideal treatment when being used with existing anticancer drugs.

### TECHNICAL FIELD

The present invention relates to an anticancer health food, and more particularly to an anticancer health food containing an extract of the mixture containing Phellinus linteus and vegetable worms. More particularly, the present invention relates to an anticancer health food prepared by using the extract of the mixture containing the Phellinus linteus and the vegetable worms at a weight ratio of 1:5 to 5:1, which can show a significantly improved anticancer effect.

### BACKGROUND ART

A malignant tumor, that is, cancer, is the worst disease threatening human health, and is the next one of the most general causes of death in Western society, second only to cardiovascular disease. In modern society, due to an increase in aging populations, an increase in the number of cigarette smokers, and air pollution, the number of lung cancer patients has significantly increased. Also, the ingestion of fatty foods following the westernization of eating habits, an increase in environmental pollutants, an increase in alcohol consumption, etc. have continuously increased the number of patients suffering a colorectal cancer, a breast cancer, a prostate cancer, etc.

Accordingly, it is urgently required to develop an anticancer material which can previously prevent and treat a cancer, thereby contributing to the improvement of human health, and the quality of healthy living.

Therapeutic agents developed for cancer treatment are nonspecific while acting on the whole body through the blood stream by general treatment injections or oral administration. Thus, they have a low specificity to a cancer cell. Accordingly, there is a disadvantage in that the administration of therapeutic agents causes many general side effects, and their side effects are more numerous than an operation or radiation therapy. Also, in the cancer treatment using conventional anticancer drugs, the drugs are administered 3 to 5 days per week, for 6 months to 1 year, at an interval of 3 to 4 weeks. This causes side effects related to the various anticancer medicines and thus there occur many cases where the treatment has to be stopped.

As described above, a conventional chemical therapeutic method for showing a medicinal effect by directly attacking a cancer cell not only causes a very strong side effect but also has non-specificity. However, in recent years, no epochal novel drug has been developed.

Accordingly, many inventors have researched natural materials having anti-cancer activity. Especially, there have been many reports on the anti-cancer activity of fungi.

At present, fungi known to have anti-cancer activity include Ganoderma lucidum, Phellinus linteus, Agaricus, vegetable worms, Gastrodia elata, Coriolus versicolor, Poria cocos, Fomitella fraxinea, Grifola frondosa, Hericium erinaceum, Fremella fuciformis, and the like.

From among the natural mushroom products, Phellinus linteus is called “Mokjiljinhuk” in Korea, and is recorded as “Sangmokyi” in the chapter “extract liquid” of Dongeuibogam. The cap has a diameter of 6 to 12 cm, and a thickness of 2 to 10 cm, and is formed into various shapes such as a half circle, a flat shape, a circular hill, a horseshoe, etc. On the surface, short dark brown hairs are densely grown, and then gradually disappear, and changed into cuticles during growth. Also, the mushroom is formed with blackish brown ring-shaped grooves, and the back splits along the width and the length. The edge is vivid yellow, the lower surface is yellowish brown, and the flesh is also yellowish brown. No stalk is formed, and a spore is light yellowish brown, and is formed into a ball shape.

The mushroom is perennial, and is a wood-rotting fungus which grows upon white mulberry trees, etc. At the initial stage, it seems to be a clumped mud lump, and then after the completion of its growth, it protrudes from a stump like a tongue. Thus, it is called ‘Soosul’ in Korea. Since polysaccharides contained in Phellinus linteus are widely known to activate an immune system and show an anti-cancer activity, an immune activity, and an anti-tumor effect, many researches have been conducted so far in Korea, Japan, China, etc. From such researches, it was found that the polysaccharide content in Phellinus linteus has an effect on anti-cancer and immunity reinforcement. Also, in Korea, Phellinus linteus is considered as a valuable drug, and is being mass-cultivated.

When Phellinus linteus is boiled down for medical use, the extracted liquid is (light) yellow and limpid, and has no taste and no scent. Phellinus linteus, itself, has a mild and plain taste, and thus is edible. It grows naturally in Korea, Japan, Australia, North America, etc.

Vegetable worms are small fungi of Ascomycota Clavicipitales Cordycipiteae, and are also called Hachodongchoong in Korea. Most of them are parasitic in an insect host and produce a fruit body in an insect carcass. The fruit body is divided into two parts, a head and a stem. The head part becomes swollen while formed into a ball shape, a cylindrical shape with both ends pointed, a scoop shape, or the like. Also, on the surface or under the skin, many perithecia are formed. Examples of an insect used as a host include Lepidoptera (Cordyceps militaris), Homoptera (C. sobolifera), Hymenoptera (C. sphecocephala), Cleoptera, Orthoptera, etc. Furthermore, some vegetable worms are parasitic in a spider host. These fungi kill the host while producing a claviform or striated fruit body. Since the vegetable worms in winter change into a mushroom in summer, they are called ‘Dongchoonghacho” in Korea. The vegetable worms have an anticancer content with a high tumor suppressing property, and thus are utilized as a good anticancer treatment supplement due to their high anticancer effect.

Up to now, medicines or functional foods, which use Phellinus linteus or vegetable worms with a high anticancer effect, have been variously developed. For example, Korean Patent Publication No. 2003-0030636 discloses a method for preparing hypertensive anticancer proteoglycan by using fermentation of a mushroom fruit body, Korean Patent Registered No. 10-0546442 discloses an anticancer composition containing an extract of Cordyceps millitaris, and Korean Patent Registered No. 10-0607907 discloses a pharmaceutical composition containing a Phellinus linteus extract, which can inhibit cancer metastasis to a liver and is excellent in immunomodulation. However, in the methods, a method for preparing any one of a Phellinus linteus extract and a vegetable worm extract, or a composition prepared by the same is known while a method for combining a Phellinus linteus extract with a vegetable worm extract, and an effect on the improvement of a physiological activity (such as an anticancer activity) according to a combination product or a combination ratio thereof is not sufficiently known.

The inventors of the present invention have continuously carried out experiments on an anticancer activity effect of a mushroom extract. As a result, they found that a mixture containing a Phellinus linteus extract and a vegetable worm extract at a weight ratio of 5:1 to 1:5 has a highly improved anticancer activity. Then, they have completed the present invention by preparing foods (such as tablets, pills, and drinks) having a high anticancer function through freeze-drying of the extract mixture, and mixing of the extract mixture with various diluting agents.

### DISCLOSURE

### Technical Problem

Therefore, the present invention has been made in view of the above-mentioned problems, and the present invention provides a health food for preventing and relieving cancer, which is prepared into tablets, pills, etc by mixing an extract of the mixture containing Phellinus linteus and vegetable worms with various diluting agents.

### Technical Solution

The anticancer health food according to the present invention includes an extract of Phellinus linteus and an extract of vegetable worms.

The health food, according to the present invention, including Phellinus linteus and vegetable worms includes an extract of the mixture containing the mycelia of Phellinus linteus and vegetable worms at a weight ratio of 1:10 to 10:1, preferably of 1:5 to 5:1.

The method for preparing the extract of the Phellinus linteus and the vegetable worms includes the steps of: mixing Phellinus linteus mycelia powder with vegetable worm mycelia powder at a predetermined weight ratio; placing the mixture powder in an extraction vessel attached with a reflux condenser; carrying out 4-hour heat-extraction of the powder three times in a water bath with a 40 to 60% ethanol aqueous solution in an amount of about 5 to 20 times by volume with respect to the weight of the powder; and vacuum-concentrating a filtrate obtained through filtering so as to provide an ethanol extract. The ethanol extract is suspended in water, and then is subjected to freeze-drying so as to provide the extract of the Phellinus linteus and the vegetable worms.

It can be understood that the extract of the Phellinus linteus and the vegetable worms according to the present invention can synergistically improve the immune activity according to the combination of its contained immunity enhancing contents. Accordingly, when the extract of the vegetable worms according to the present invention is used as a food, the extract as it is may be added or may be used together with another food or other food contents. Also, the extract may be appropriately used according to a conventional method.

The amount of an active component with respect to the total mixture may be appropriately determined according to the usage purpose (prevention, health, or therapeutic treatment), and may be based on an effective dose of the pharmaceutical composition. For a long-time ingestion with a purpose of promoting health, hygiene or health adjustment, the amount of the active component may be less than the above described range. Otherwise, since the active component has no problem in view of safety, the amount may be greater than the above described range.

There is no specific limitation in the kind of the food. Examples of a food to be added with the component may include meat, sausage, bread, chocolate, candies, snack, sweets, pizza, ramen, other noodles, gums, dairy foods such as ice cream, various kinds of soups, drinks, tea, drink preparations, alcohol drinks, and vitamin complexes.

Hereinafter, the present invention will be described in detail with reference to Examples.

However, the following examples are only for illustrative purposes and are not intended to limit the scope of the invention.

### Advantageous Effects

From among basidiomycetes, Phellinus linteus has the best anticancer effect. The anticancer effect is mainly caused from protein-bound saccharides or polysaccharides, basic proteins, etc. The polysaccharides contained in the Phellinus linteus activate the immune system, thereby causing an anticancer activity and an immune activating anti-tumor effect. Also, vegetable worms have a high anticancer effect like the Phellinus linteus. Their anticancer component is produced during their growth, which causes no side effect at all while improving a resisting force and having a highly therapeutic effect on infection of bacteria or virus.

In the present invention, since extracts of Phellinus linteus mycelia and vegetable worm mycelia are mixed with each other, respective polysaccharides of the two mushrooms synergically improve the immune activity. This increases or activates immune systems which recognize abnormal cancer cells as antigens and distinguish them from normal cells. Thus, there is an effect in that continuous division and proliferation of cancer cells can be inhibited.

Accordingly, when the extract is used in parallel with a conventional anticancer medicine, it is possible to achieve an ideal therapeutic effect. Especially, the inventive extract can show a very high effect in treatment and prevention of a lung cancer, a liver cancer, and a breast cancer. Also, unlike a conventional anticancer medicine causing a side effect such as serious loss of hair, peripheral neuropathy, myalgia, the inventive anticancer health food can be safely ingested by anybody with no side effect because it is obtained by using, as raw materials, Phellinus linteus and vegetable worms (officially approved as breeds suitable as food raw materials, by the Korea Food and Drug Administration).

### BRIEF DESCRIPTION OF THE DRAWINGS

The foregoing and other objects, features and advantages of the present invention will become more apparent from the following detailed description when taken in conjunction with the accompanying drawing in which:

FIG. 1 is a view of the measurement of a tumor volume inhibiting ratio and a tumor weight inhibiting ratio when a lung cancer cell (Lewis Lung Carcinoma: LLC) was transplanted to a mouse, and then on the 14^{th} day, the administration of a sample (the inventive extract and Taxol) was stopped.

### BEST MODE

### Mode for Invention

Hereinafter, preferred exemplary embodiments of the present invention will be described. However, the present invention is not limited by the Examples below, but may be realized in other forms. The Examples as described below are provided to thoroughly and completely disclose the invention and fully convey the scope of the invention to a person skilled in the art.

First, the health food according to the present invention includes a mixture containing a Phellinus linteus extract and a vegetable worm extract at a weight ratio of 1:10 to 10:1, preferably of 1:5 to 5:1.

### Example 1

### An Extract of a 1:1 Mixture of Phellinus linteus and Vegetable Worms

Phellinus linteus mycelia powder was mixed with vegetable worm mycelia powder at a 1:1 weight ratio, and then the mixture powder was placed in an extraction vessel attached with a reflux condenser. Then, the mixture was subjected to 4-hour heat-extraction three times in a water bath with a 50% ethanol aqueous solution in an amount of 10 times by weight with respect to the weight of the powder. Then, a filtrate obtained through filtering was vacuum-concentrated so as to provide an ethanol extract. The ethanol extract was suspended in water, and then is subjected to freeze-drying so as to provide the extract of the mixed powder of the Phellinus linteus and the vegetable worms.

### Example 2

### An Extract of a 1:3 Mixture of Phellinus linteus and Vegetable Worms

The extract of the mixture of the Phellinus linteus and the vegetable worms was prepared in the same manner as described in Example 1 except that Phellinus linteus mycelia powder was mixed with vegetable worm mycelia powder at a 1:3 weight ratio.

### Example 3

### An Extract of a 3:1 Mixture of Phellinus linteus and Vegetable Worms

The extract of the mixture of the Phellinus linteus and the vegetable worms was prepared in the same manner as described in Example 1 except that Phellinus linteus mycelia powder was mixed with vegetable worm mycelia powder at a 3:1 weight ratio.

### Example 4

### An Extract of a 1:5 Mixture of Phellinus linteus and Vegetable Worms

The extract of the mixture of the Phellinus linteus and the vegetable worms was prepared in the same manner as described in Example 1 except that Phellinus linteus mycelia powder was mixed with vegetable worm mycelia powder at a 1:5 weight ratio.

### Example 5

### An Extract of a 5:1 Mixture of Phellinus linteus and Vegetable Worms

The extract of the mixture of the Phellinus linteus and the vegetable worms was prepared in the same manner as described in Example 1 except that Phellinus linteus mycelia powder was mixed with vegetable worm mycelia powder at a 5:1 weight ratio.

### Example 6

### An Extract of a 1:10 Mixture of Phellinus linteus and Vegetable Worms

The extract of the mixture of the Phellinus linteus and the vegetable worms was prepared in the same manner as described in Example 1 except that Phellinus linteus mycelia powder was mixed with vegetable worm mycelia powder at a 1:10 weight ratio.

### Example 7

### An Extract of a 10:1 Mixture of Phellinus linteus and Vegetable worms

The extract of the mixture of the Phellinus linteus and the vegetable worms was prepared in the same manner as described in Example 1 except that Phellinus linteus mycelia powder was mixed with vegetable worm mycelia powder at a 10:1 weight ratio.

### Comparative Example 1

### Taxol

Taxol was bought from SIGMA(PO115).

### Experimental Example 1

### Test on an Anticancer Effect of the Prepared Mixture Extract of Phellinus linteus and Vegetable Worms

In order to test an anticancer effect of the mixture extract of Phellinus linteus and vegetable worms, from Examples 1 to 7, and Comparative 1, an animal test was carried out. A mouse used for the experiment was bought from Jung Ang Lab. Animal Inc. (black, Japan, BDF1 male, weight: 20±2 g). The breeding temperature was 22±2° C., and solid feed stuff (Samyang feed Co.) and water were sufficiently supplied. In each group, 7 mice were used. Except for 2 mice with high standard deviations, the results of 5 mice were noted in the data.

A solvent for the extraction was an extra pure reagent (E.P.). For the test sample, a sterilized saline solution manufactured by Daihan Pharm Co. Ltd. was used. For the culture of a cancer cell, an RPMI 1640 medium (WelGENE Inc.) was used, and Trypsin-EDTA was manufactured by GIBCO. Besides, a test tube required for cell culture, a culture flask, and a filter for obtaining a cancer cell were manufactured by Falcon. Devices used for cancer transplantation were disinfected at 120° C. for 30 min, and a cancer volume was measured by a Digital Caliper.

A lung cancer cell (Lewis Lung Carcinoma: LLC) used for the experiment was distributed from the Korea Research Institute of Bioscience and Biotechnology. The lung cancer cell (Lewis Lung Carcinoma: LLC) was cultured by RPMI 1640 medium added with 10% FBS in a 37° C. incubator, and for the detachment from an attachment surface, Trypsin-EDTA was used. When the cells are filled up in a 200 ml culture flask, they were transplanted to two animals. After the cells were grown in vivo, they were used for an animal test.

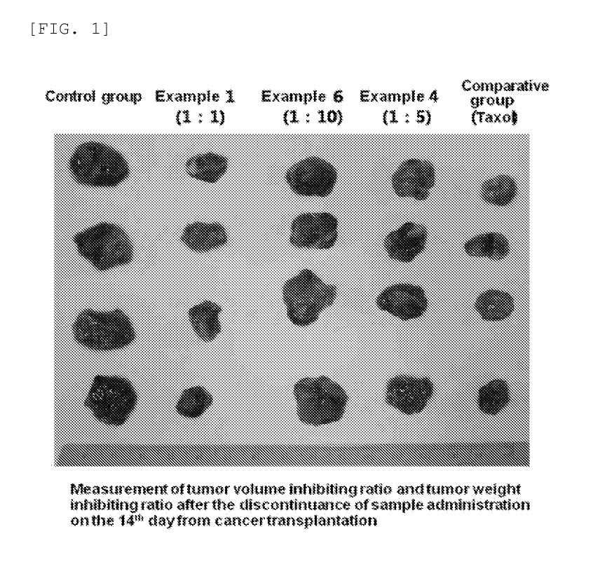

In an anticancer animal test, a tumor grown up to a size of about 15×15×15 mm³ was separated, and a cancer cell was separated from other cells by using a filer (Falcon). Then, a cancer cell which was washed several times and had a concentration of 2×10⁵ cell/ml was then injected in a quantity of 0.1 ml to an armpit of a test animal so as to generate a cancer. From the day after the cancer transplantation, for 14 days, powder obtained by mixing Phellinus linteus and vegetable worms at a weight ratio of 1:1 (Example 1), 1:3(Example 2), 3:1(Example 3), 1:5(Example 4), 5:1(Example 5), 1:10(Example 6), 10:1(Example 7) was orally administered daily to a black mouse (Japan) in an amount of 200 mg/kg. Meanwhile, as a positive control group, Taxol was intraperitoneally injected daily in an amount of 50 mg/kg once a day for 14 days. After 14 days, the administration of the extract powder of Phellinus linteus and vegetable worms was stopped, and a Digital Caliper was used to measure the results. As shown in FIG. 1, a tumor volume (TV) was calculated by Length (mm)×Width²/2, and an inhibition ratio of a tumor volume (IRTV) was calculated by (TV of control−TV of test)×100/TV of control. After the measurement of a tumor volume, a tumor lump was obtained through an operation and its weight was measured. An inhibition ratio of tumor weight (IRTW) was calculated by (TW of control−TW of test)×100/TW of control.

All data were indicated by mean±S.D., and the analysis was carried out by Anova test. Also, when p values are 0.05 or less, they were determined to have significance. The test results are noted in Table 1 below.

TABLE 1
Test results of an anticancer effect of the prepared mixture
powder of Phellinus linteus and vegetable worms

| | Administration | | Tumor volume | | Tumor weight |
|---|---|---|---|---|---|
| | amount | Tumor size | inhibiting | Tumor weight | inhibiting |
| index | (mg/kg) | (mm³) | ratio (%) | (g) | ratio (%) |
| Control group | 0 | 1985 ± 233 | 0 | 1.73 ± 0.3 | 0 |
| Example 1 (1:1) | Phellinus linteus 100 | 765 ± 198 | 61 | 0.63 ± 0.4 | 63 |
| | vegetable worms 100 | | | | |
| Example 2 (1:3) | Phellinus linteus 50 | 887 ± 178 | 55 | 0.78 ± 0.5 | 54 |
| | vegetable worms 150 | | | | |
| Example 3 (1:3) | Phellinus linteus 150 | 850 ± 154 | 57 | 0.76 ± 0.1 | 56 |
| | vegetable worms 50 | | | | |
| Example 4 (1:5) | Phellinus linteus 34 | 1007 ± 139 | 45 | 0.93 ± 0.3 | 46 |
| | vegetable worms 166 | | | | |
| Example 5 (5:1) | Phellinus linteus 166 | 1048 ± 121 | 47 | 0.91 ± 0.5 | 47 |
| | vegetable worms 34 | | | | |
| Example 6 (1:10) | Phellinus linteus 18 | 1678 ± 145 | 15 | 1.43 ± 0.1 | 17 |
| | vegetable worms 192 | | | | |
| Example 7 (10:1) | Phellinus linteus 192 | 1561 ± 132 | 21 | 1.40 ± 0.2 | 19 |
| | vegetable worms 18 | | | | |
| Taxol (positive | 50 | 649 ± 182 | 67 | 0.61 ± 0.3 | 64 |
| control group) | | | | | |

In Table 1, the results of an anticancer effect of powder obtained by mixing Phellinus linteus and vegetable worms at a weight ratio of 1:1 (Example 1), 1:3(Example 2), 3:1(Example 3), 1:5(Example 4), 5:1(Example 5), 1:10(Example 6), 10:1(Example 7), and an anticancer effect of Taxol (Comparative Example 1) are noted. 50 mg/kg of Taxol, which is being clinically widely used, showed a tumor volume inhibiting ratio of 67%, and a tumor weight inhibiting ratio of 64%. Meanwhile, the inventive extract of Phellinus linteus and vegetable worms at a mixing ratio of 1:1 (Example 1) showed a tumor volume inhibiting ratio of 61%, and a tumor weight inhibiting ratio of 63%. Thus, it was found that a 1:1 mixing ratio showed the highest anticancer effect. Also, an extract of a mixture containing Phellinus linteus and vegetable worms at weight ratios of 1:3 (Example 2) and 3:1 (Example 3) showed tumor volume/weight inhibiting ratios of 50 to 60%, respectively, and an extract of a mixture containing Phellinus linteus and vegetable worms at weight ratios of 1:5 (Example 4) and 5:1 (Example 5) showed tumor volume/weight inhibiting ratios of 40 to 50%, respectively. It was determined that the extracts showed a surprisingly high anticancer effect in consideration of the fact that these are mushroom extracts.

From the results, it can be seen that although a mixture powder of extracts of Phellinus linteus and vegetable worms has an anticancer effect slightly lower than Taxol, it can show a sufficient effect on cancer chemical prevention or cancer inhibition through ingestion at regular times.

### Usage Example 1

### Preparation of an Anticancer Tablet

100 g of the mixture powder of extracts of Phellinus linteus and vegetable worms, obtained from Example 1, was mixed with 70 g of lactose, 75 g of potato starch, and 10 g of colloidal silicic acid. The mixture was added with a 10% gelatin solution, pulverized, filtered by 14 meshes, and dried. Then, 65 g of potato starch, 25 g of talc, and 2 g of magnesium stearate were added thereto, and mixed. Then, the resultant mixture was tabletted in such a manner that one tablet may have a weight of 400 mg.

### Usage Example 2

### Preparation of an Anticancer Capsule

60 wt % of the mixture powder of extracts of Phellinus linteus and vegetable worms, obtained from Example 1, was mixed with 15 wt % of jujube powder and 25 wt % of crystalline cellulose by using 70% alcohol. The mixture was dried at 60° C. for 30 hours, pulverized, filtered by 50 meshes, and filled in a capsule. In the manufacturing of the capsule, a unit amount was 500 mg/cap.

### Usage Example 3

### Preparation of an Anticancer Drink

0.48 to 1.28 mg of the mixture powder of extracts of Phellinus linteus and vegetable worms, obtained from Example 1, 522 mg of honey, 5 mg of thioctic acid amid, 10 mg of nicotinic acid amide, 3 mg of riboflavin sodium hydrochloric acid, 2 mg of pyridoxine hydrochloride, 30 mg of inositol, 50 mg of orotic acid, and 200 ml of water were used to manufacture a drink according to a conventional method.

1. (canceled)

2. A health food for inhibiting or treating cancer, comprising, as an active ingredient, an ethanol aqueous solution extract of a mixture containing mycelia of Phellinus linteus and vegetable worms in a weight ratio of about from 1:5 to about 5:1.

3. The health food as claimed in claim 2, wherein the ethanol aqueous solution extract contains a 40 to 60% ethanol aqueous solution extract as an active ingredient.

4. The health food as claimed in claim 2, which is prepared into a tablet, a capsule, or a drink.

5. The health food as claimed in claim 3, which is prepared into a tablet, a capsule, or a drink.

6. A method for preparing an extract of a mixture of mycelia of Phellinus linteus and vegetable worms Phellinus, comprising
a) mixing Phellinus linteus mycelia powder with vegetable worm mycelia powder
b) carrying out heat-extraction of the mixture obtained from step a) for about 4 hours three times in a water bath comprising 40 to 60% ethanol aqueous solution in an amount of about 5 to 20 times by volume with respect to the weight of the mixture;
c) filtering a resultant from the heat-extraction in step b); and
d) conducting vacuum-concentration of a filtrate from step c).

7. A method for inhibiting or treating cancer comprising administering a subject the health food of claim 2.
